(19) United States
(12) Patent Application Publication
(10) Pub. No.: US 2023/0057553 A1
(43) Pub. Date: Feb. 23, 2023

(54) REPLACEMENT HEART VALVE IMPLANT

(71) Applicant: BOSTON SCIENTIFIC SCIMED, INC., MAPLE GROVE, MN (US)

(72) Inventors: Michael G. Hayes, Galway (IE); Andrea Goldbey, Galway (IE); Tara Hogan, Galway (IE)

(73) Assignee: BOSTON SCIENTIFIC SCIMED, INC., MAPLE GROVE, MN (US)

(21) Appl. No.: 17/889,774

(22) Filed: Aug. 17, 2022

Related U.S. Application Data
(60) Provisional application No. 63/234,580, filed on Aug. 18, 2021.

(51) Int. Cl. A61F 2/24

(52) U.S. Cl. CPC A61F 2/2418; A61F 2/2427; A61F 2220/0075

(57) ABSTRACT

Example heart valves are disclosed. An example heart valve may comprise an expandable framework configured to shift from a closed configuration to an expanded configuration, the expandable framework having an inflow end, an outflow end and a lumen extending therein. The heart valve may also include a plurality of valve leaflets coupled to the expandable framework, each of the plurality of valve leaflets including a root edge and a free edge. Further, the heart valve may also include an outer skirt coupled to the plurality of valve leaflets, the outer skirt including a proximal edge, wherein the root edge of each of the plurality of valve leaflets is attached to the proximal edge of the outer skirt.

### CROSS-REFERENCE TO RELATED APPLICATIONS

This application claims the benefit of priority of U.S. Provisional Application No. 63/234,580 filed Aug. 18, 2021, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a replacement heart valve implant.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

An example heart valve may comprise an expandable framework configured to shift from a closed configuration to an expanded configuration, the expandable framework having an inflow end, an outflow end and a lumen extending therein. The heart valve may also include a plurality of valve leaflets coupled to the expandable framework, each of the plurality of valve leaflets including a root edge and a free edge. Further, the heart valve may also include an outer skirt coupled to the plurality of valve leaflets, the outer skirt including a proximal edge, wherein the root edge of each of the plurality of valve leaflets is attached to the proximal edge of the outer skirt.

In addition or alternatively to any example described herein, wherein the outer skirt is positioned along an outer surface of the expandable framework.

In addition or alternatively to any example described herein, wherein the plurality of valve leaflets is positioned within the lumen of the expandable framework.

In addition or alternatively to any example described herein, wherein the root edge of each of the plurality of valve leaflets is sutured to the proximal edge of the outer skirt.

In addition or alternatively to any example described herein, wherein root edge of each of the plurality of valve leaflets and the proximal edge of the outer skirt are sutured around the expandable framework such that the root edge of the plurality of valve leaflets and the proximal edge of the outer skirt may move relative to the expandable framework.

In addition or alternatively to any example described herein, wherein the outer skirt further includes a distal edge, and wherein the distal edge of the outer skirt is attached to one or more of the plurality of valve leaflets at one or more attachment points.

In addition or alternatively to any example described herein, wherein the outer skirt extends radially outward from the expandable framework to capture fluid flow around an exterior of the expandable framework within the outer skirt.

In addition or alternatively to any example described herein, wherein the outer skirt is substantially impervious to fluid.

In addition or alternatively to any example described herein, wherein the proximal edge of the outer skirt is disposed upstream of the inflow end of the expandable framework.

In addition or alternatively to any example described herein, wherein the expandable framework further includes an upper crown, wherein the distal edge of the outer skirt is disposed proximal of the upper crown.

Another heart valve may comprise an expandable framework having an inflow end, an outflow end, and a medial region positioned between the inflow end and the outflow end. The heart valve may also include a plurality of valve leaflets attached to the medial region of the expandable framework, each of the plurality of valve leaflets including a root edge. Further, the heart valve may include an outer skirt disposed along an outer surface of the expandable framework, the outer skirt including a proximal edge and a distal edge. Further, the outer skirt is configured to shift between an unexpanded configuration and an expanded configuration and the root edge of each of the plurality of valve leaflets is attached to the proximal edge of the outer skirt.

In addition or alternatively to any example described herein, wherein the plurality of valve leaflets is positioned within a lumen of the expandable framework.

In addition or alternatively to any example described herein, wherein the root edge of each of the plurality of valve leaflets is sutured to the proximal edge of the outer skirt.

In addition or alternatively to any example described herein, wherein root edge of each of the plurality of valve leaflets and the proximal edge of the outer skirt are sutured around the expandable framework such that the root edge of the plurality of valve leaflets and the proximal edge of the outer skirt may move relative to the expandable framework.

In addition or alternatively to any example described herein, wherein the distal edge of the outer skirt is attached to one or more of the plurality of valve leaflets at one or more attachment points.

In addition or alternatively to any example described herein, wherein the outer skirt is configured to capture fluid flow around an exterior of the expandable framework within the outer skirt.

In addition or alternatively to any example described herein, wherein the outer skirt is substantially impervious to fluid.

In addition or alternatively to any example described herein, wherein the proximal edge of the outer skirt is disposed upstream of the inflow end of the expandable framework.

In addition or alternatively to any example described herein, wherein the expandable framework further includes an upper crown, wherein the distal edge of the outer skirt is disposed proximal of the upper crown.

An example method for delivering a heart valve implant at a target site includes advancing a heart valve implant to a target site adjacent the heart, the heart valve implant including: an expandable framework configured to shift from a closed configuration to an expanded configuration, the expandable framework having an inflow end, an outflow end and a lumen extending therein. The heart valve also includes a plurality of valve leaflets coupled to the expandable framework, each of the plurality of valve leaflets including a root edge and a free edge. Further, the heart valve also includes an outer skirt coupled to the plurality of valve leaflets, the outer skirt including a proximal edge, wherein the root edge of each of the plurality of valve leaflets is attached to the proximal edge of the outer skirt. Further, the method also includes deploying the implantable heart valve at the target site.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example medical device system in a delivery configuration;

FIG. 2 illustrates the example medical device system of FIG. 1 in a deployed configuration;

FIG. 3 illustrates an example replacement heart valve implant positioned within a portion of a heart;

FIG. 4 illustrates an example replacement heart valve implant in a first configuration;

FIG. 5 illustrates the replacement heart valve implant of FIG. 4 in a second configuration.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses other challenges in that the treatment often requires the repair or outright replacement of the defective heart valve. Such therapies may be highly invasive to the patient. Disclosed herein is an apparatus, system, and/or method that may be used for preparing and/or delivering a medical implant to a portion of the cardiovascular system in order to diagnose, treat, and/or repair the system. In some embodiments, the apparatus, system, and/or method disclosed herein may be used before and/or during a procedure to diagnose, treat, and/or repair a defective heart valve (e.g., the aortic valve, the mitral valve, etc.). In addition, a replacement heart valve implant may be delivered percutaneously and thus may be much less invasive to the patient. The apparatus, system, and/or method disclosed herein may also provide other desirable features and/or benefits as described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the strut”, or other features may be equally referred to all instances and quantities beyond one of said feature. As such, it will be understood that the following discussion may apply equally to any and/or all of the components for which there are more than one within the replacement heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems, devices, and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient's anatomy, such as but not limited to, arteries, veins, and/or other body lumens.

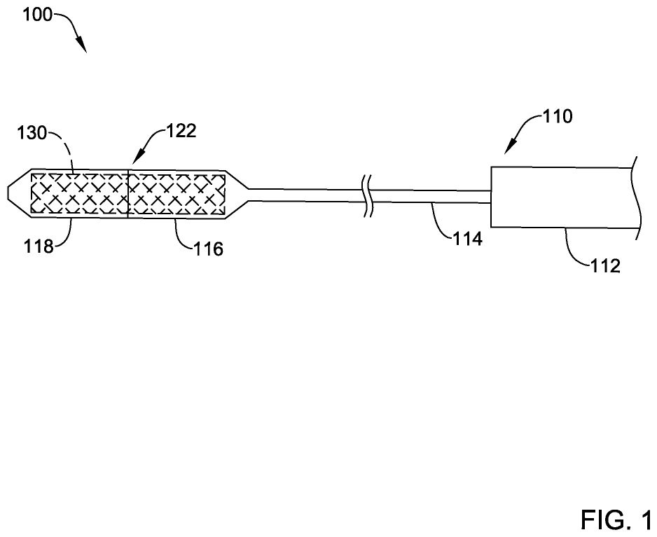

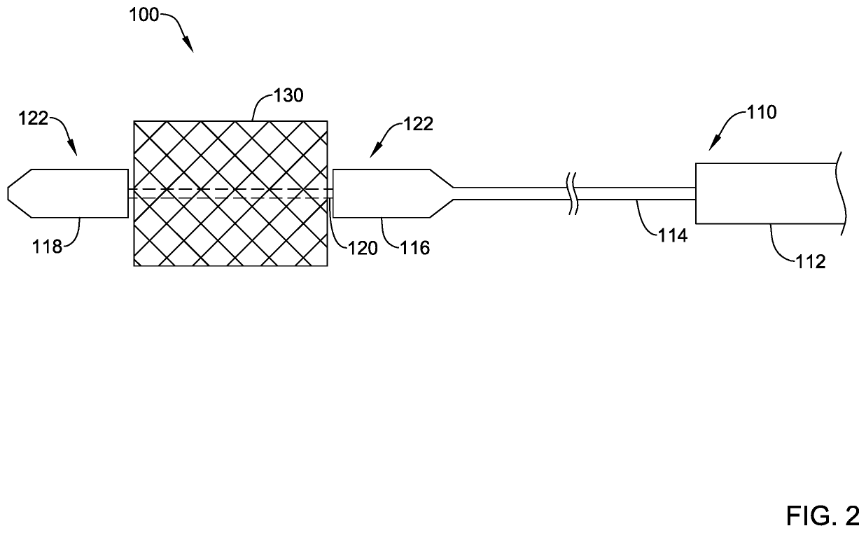

A medical device system 100, as illustrated in FIG. 1 for example, may generally be described as a catheter system that includes an implant delivery device 110 for delivering a replacement heart valve implant 130 which may be coupled to the implant delivery device 110 and disposed within a lumen of the implant delivery device 110 during delivery of the replacement heart valve implant 130. The implant delivery device 110 may include a proximal handle 112 and an elongate shaft 114 extending distally from the proximal handle 112. In some embodiments, the implant delivery device 110 and/or the elongate shaft 114 may include a proximal sheath 116 and a distal sheath 118. The implant delivery device 110 may include an inner shaft 120 (shown in FIG. 2) slidably disposed within a lumen of the elongate shaft 114. The inner shaft 120 may be fixedly attached to the distal sheath 118. In some embodiments, the inner shaft 120 may include a guidewire lumen extending therethrough. In some embodiments, the proximal handle 112 may be configured to manipulate and/or translate the proximal sheath 116 and/or the distal sheath 118 relative to each other. In some embodiments, the proximal handle 112 may be configured to manipulate and/or translate the inner shaft 120 relative to the elongate shaft 114 and/or the proximal sheath 116.

During delivery of the replacement heart valve implant 130, the replacement heart valve implant 130 may be disposed within the proximal sheath 116 and/or the distal sheath 118 in a collapsed configuration, as illustrated in FIG. 1. In some embodiments, the proximal sheath 116 and/or the distal sheath 118 may collectively define a stent holding portion 122 of the implant delivery device 110. In some embodiments, the stent holding portion 122 may be configured to constrain the replacement heart valve implant 130 in the collapsed configuration. In some embodiments, the replacement heart valve implant 130, the replacement heart valve implant 130 may be releasably coupled to the inner shaft 120.

In use, the medical device system 100 may be advanced percutaneously through the vasculature to a position adjacent to a treatment site. For example, the medical device system 100 may be advanced through the vasculature and across the aortic arch to a position adjacent to a defective aortic valve. Alternative approaches to treat a defective aortic valve and/or other heart valve(s) are also contemplated with the medical device system 100. After navigating the implant delivery device 110 and/or the stent holding portion 122 to the treatment site, the proximal sheath 116 and/or the distal sheath 118 may be translated relative to each other to open the stent holding portion 122. When unconstrained by the stent holding portion 122, the replacement heart valve implant 130 may be configured to shift from the collapsed configuration to an expanded configuration, as illustrated in FIG. 2. In at least some interventions, the replacement heart valve implant 130 may be deployed within the native valve (e.g., the native valve is left in place and not excised). Alternatively, the native valve may be removed (such as through valvuloplasty, for example) and the replacement heart valve implant 130 may be deployed in its place as a replacement. Some suitable but non-limiting materials for the medical device system 100, implant delivery device 110, the proximal handle 112, the elongate shaft 114, the proximal sheath 116, the distal sheath 118, the inner shaft 120, the stent holding portion 122, and/or components or elements thereof, for example metallic materials and/or polymeric materials, are described below.

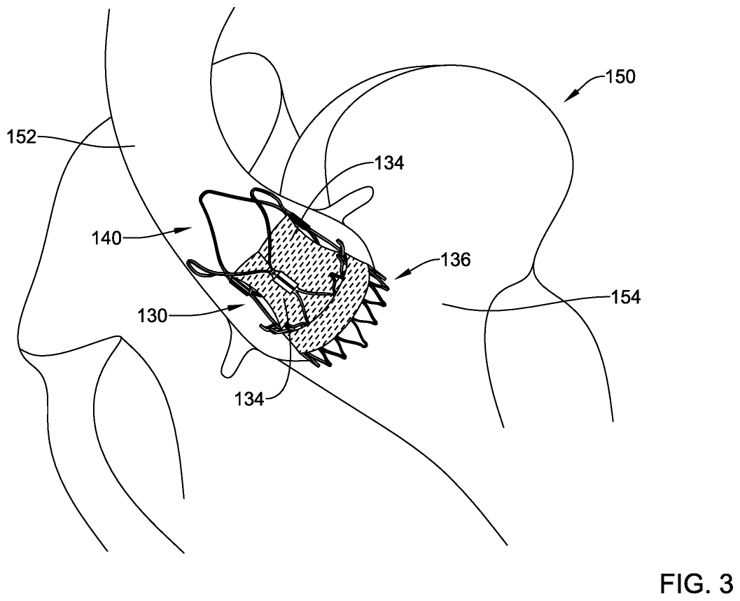

FIG. 3 illustrates the replacement heart valve implant 130 deployed within an example heart 150. As described herein, the medical device system 100 may be advanced percutaneously through the vasculature and across the aortic arch 152 to a position adjacent a treatment site. After being advanced to a position adjacent a treatment site, the medical device system 100 may be manipulated such that the replacement heart valve implant 130 shifts from a collapsed configuration (e.g., delivery configuration) to an expanded configuration (e.g., deployed configuration).

As will be described in greater detail herein, FIG. 3 illustrates that the replacement heart valve implant may include a first end including a lower crown 136 and a second end including a plurality of stabilization arches 140. In some embodiments, the lower crown 136 may be disposed at and/or may correspond to the inflow end of the replacement heart valve implant 130. Additionally, in some embodiments, the plurality of stabilization arches 140 may be disposed at and/or may correspond to the outflow end of the replacement heart valve implant 130. Additionally, FIG. 3 illustrates that the replacement heart valve implant 130 may include one or more valve leaflets 134 positioned in a central region along the longitudinal axis of the replacement heart valve implant 130.

In some embodiments, the plurality of stabilization arches 140 of the replacement heart valve implant 130 may extend downstream in a direction opposite the lower crown 136. In other words, it can be appreciated that as the heart pumps fluid (e.g., blood) from the left ventricle 154 into the aorta arch 152, the fluid may first flow through lower crown 136, then through the one or more valve leaflets 134 and lastly through the plurality of stabilization arches 140.

As will be described in greater detail below, FIG. 3 illustrates that the replacement heart valve implant 130 may include a plurality of valve leaflets 134 disposed within a central lumen of the replacement heart valve implant 130. It can be appreciated that as fluid flows from the left ventricle 154 to the aorta 152, it may pass through the one or more valve leaflets 134. It can be further appreciated that the valve leaflets 134, together, are designed to shift between a first (e.g., closed) configuration and a second (e.g., open) configuration, thereby permitting the fluid to flow in a direction from the left ventricle 154 to the aorta 152 while preventing fluid from flowing from the aorta 152 back into the left ventricle 154.

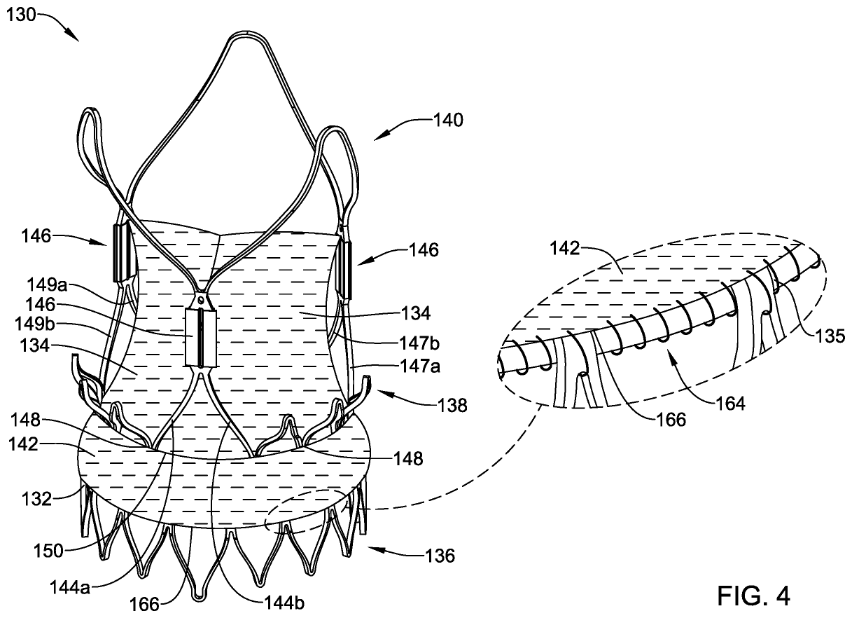

FIG. 4 illustrates an example replacement heart valve implant 130. It can be appreciated that the replacement heart valve implant 130 can be any type of heart valve (e.g., a mitral valve, an aortic valve, etc.). In use, the replacement heart valve implant 130 can be implanted (e.g., surgically or through transcatheter delivery) in a mammalian heart. As described herein, the replacement heart valve implant 130 can be configured to allow one-way flow through the replacement heart valve implant 130 from an inflow end to an outflow end.

The replacement heart valve implant 130 may include an expandable framework 132 defining a central lumen which, in some embodiments, may be substantially cylindrical. The side of the expandable framework 132 and other components facing the central lumen can be referred to as the luminal surface or luminal side. The opposite or outer side of the expandable framework 132 and other components (e.g., facing away from the central lumen) can be referred to as the abluminal surface or abluminal side. In some embodiments, the expandable framework 132 may have a substantially circular cross-section. In some embodiments, the expandable framework 132 can have a non-circular (e.g., D-shaped, elliptical, etc.) cross-section. In some embodiments, a non-circular expandable framework can be used to repair an aortic valve or another non-circular valve in the patient's heart or body. Some suitable but non-limiting examples of materials that may be used to form the expandable framework 132, including but not limited to metals and metal alloys, composites, ceramics, polymers, and the like, are described below.

As discussed herein, the expandable framework 132 may be configured to shift from a collapsed configuration to an expanded configuration. In some embodiments, the expandable framework 132 may be self-expanding. In some embodiments, the expandable framework 132 may be self-biased toward the expanded configuration. In some embodiments, the expandable framework 132 may be mechanically expandable. In some embodiments, the expandable framework 132 may be balloon expandable. Other configurations are also contemplated.

As illustrated in FIG. 4, the expandable framework 132 may include a plurality of frame struts. The frame struts may define a framework or lattice structure. In some embodiments, the plurality of frame struts may define a plurality of interstices (e.g., openings) between adjacent frame struts and/or through the expandable framework 132 from the luminal side to the abluminal side.

In some embodiments, the expandable framework 132 and/or the plurality of frame struts may define a lower crown 136, an upper crown 138, and a plurality of stabilization arches 140. In some embodiments, the lower crown 136 may be disposed at and/or may correspond to the inflow end of the expandable framework 132 and/or the replacement heart valve implant 130. In some embodiments, the upper crown 138 and/or the plurality of stabilization arches 140 may be disposed at and/or may correspond to the outflow end of the expandable framework 132 and/or the replacement heart valve implant 130. In some embodiments, the plurality of stabilization arches 140 may extend downstream of and/or away from the upper crown 138 in a direction opposite the lower crown 136. In some embodiments, the upper crown 138 may be disposed longitudinally and/or axially between the lower crown 136 and the plurality of stabilization arches 140.

Additionally, FIG. 4 illustrates that the replacement heart valve implant 130 may include a plurality of valve leaflets 134 disposed within the central lumen. In some embodiments, the plurality of valve leaflets 134 may be comprised of a polymer, such as a thermoplastic polymer. In some embodiments, the plurality of valve leaflets 134 may include at least 50 percent by weight of a polymer. In some embodiments, the plurality of valve leaflets 134 may be formed from bovine pericardial or other living tissue. Other configurations and/or materials are also contemplated.

The plurality of valve leaflets 134 may be secured to the expandable framework 132. For example, each of the plurality of valve leaflets 134 may include a “root edge” coupled to the expandable framework 132 and a free edge (e.g., a coaptation edge) movable relative to the root edge to coapt with the coaptation edges of the other leaflets along a coaptation region. It can be appreciated that the root edge of each of the plurality of valve leaflets 134 may be defined as the edge of a valve leaflet 134 that is opposite to the free edge (e.g., coaptation edge) of the valve leaflet 134.

In some embodiments, the plurality of valve leaflets 134 may be integrally formed with each other, such that the plurality of valve leaflets 134 is formed as a single unitary and/or monolithic unit. As will be described in greater detail below, in some instances, the plurality of valve leaflets 134 may be formed integrally and/or coupled with other structures, such as an outer skirt 142. In can be appreciated that, for embodiments in which the replacement heart valve implant 130 includes a plurality of leaflets 134 coupled to an additional structure (e.g., the outer skirt 142 shown in FIG. 4), the root edge of each of the plurality of valve leaflets 134 may define the location along each valve leaflet 134 opposite the free edge (e.g., coaptation edge) where each of the plurality of valve leaflets 134 is coupled to those other structures (e.g., the outer skirt 142 shown in FIG. 4). The method of coupling the root edge of each of the plurality of valve leaflets 134 with a lower edge of the outer shirt 142 will be described in greater detail below.

As described herein, the free edges of the plurality of valve leaflets 134 may move into coaptation with one another in a closed position to substantially restrict fluid from flowing back into the left ventricle 154 after passing through the valve leaflets 134 and into the aorta 152. Specifically, the plurality of valve leaflets 134 may coapt to fill up or close the central lumen of the replacement heart valve implant 130 thereby impeding the flow of fluid in an upstream or retrograde direction. The free edges of the plurality of valve leaflets 134 may be move apart from each other in an open position to permit fluid flow through the replacement heart valve implant 130. Specifically, the plurality of valve leaflets 134 may move apart from each other to open the central lumen of the replacement heart valve implant 130 thereby permitting the flow of fluid in a downstream or antegrade direction (e.g., the plurality of valve leaflets 134 may move apart from each other as the heart pumps blood from the left ventricle 152 into the aorta 152). In FIG. 4, the plurality of valve leaflets 134 is shown in the closed position.

As illustrated in FIG. 4, the free edge of each valve leaflet 134 may include one or more connection portions which are coupled to the expandable framework 132 at a plurality of commissures 146. For example, FIG. 4 shows that the plurality of valve leaflets 134 may be secured and/or attached to the expandable framework 132 along the luminal side of each of the plurality of commissures 146. In other words, the free edge of each of the plurality of valve leaflets 134 may extend between the plurality of commissures 146, whereby a portion of the free edge may be coupled to the luminal side of the plurality of commissures 146. In some embodiments, the plurality of valve leaflets 134 and/or the connection portions may be attached to the expandable framework 132 using sutures, adhesives, or other suitable methods.

In some embodiments, the plurality of commissures 146 may be disposed at a base of the plurality of stabilization arches 140. In some embodiments, each of the plurality of commissures 146 may connect circumferentially adjacent stabilization arches of the plurality of stabilization arches 140. In some embodiments, the plurality of commissures 146 may be disposed longitudinally between the plurality of stabilization arches 140 and the upper crown 138. In some embodiments, the plurality of commissures 146 may be disposed distal of the plurality of stabilization arches 140 and proximal of the upper crown 138. In at least some embodiments, between circumferentially adjacent commissures of the plurality of commissures 146, the replacement heart valve implant 130 may be devoid of the expandable framework 132 at a longitudinal position radially outward of the free edges of the plurality of valve leaflets 134. As such, the free edges of the plurality of valve leaflets 134 may be free from direct contact with the expandable framework 132 as the plurality of valve leaflets 134 opens and/or closes.

Additionally, FIG. 4 illustrates that the expandable framework 132 may include a plurality of interleaflet struts 144a/144b/147a/147b/149a/149b positioned proximal (e.g., upstream) of the plurality of commissures 146. It can be appreciated that one pair of interleaflet struts may extend proximally from each of the three commissures 146 illustrated in FIG. 4. For example, the pair of interleaflet struts 144a/144b extends proximally from a single commissure 146, the pair of interleaflet struts 147a/147b extends proximally from another single commissure 146, and the pair of interleaflet struts 149a/149b extends proximally from another single commissure 146.

Additionally, it can be appreciated that each of the interleaflet struts may integrate with the framework struts defining the lower crown 136. For example, the proximal end regions of each of the interleaflet struts 144a/144b/147a/147b/149a/149b may form the distal end region of a portion of the struts defining the lower crown 136 (it is noted that the proximal end regions of the interleaflet struts are hidden by the outer skirt 142 in FIG. 4). In some instances, each “pair’ of interleaflet struts, in combination with a portion of the struts of the lower crown 136 may be referred to as an “interleaflet triangle.” The interleaflet triangle may be defined as a portion of the expandable framework 132 which forms a substantially triangular-shaped free-cell, interstitial space.

FIG. 4 further illustrates that, in some instances, most of the outer facing surface of each of the plurality of valve leaflets 134 may not be in direct contact with the luminal surface of the expandable framework. In other words, while a portion of the free edges of the plurality of valve leaflets 134 and the root edge of the plurality of valve leaflets 134 may be coupled to a portion of the expandable framework 132 (e.g. the commissures 146 and the lower edge of the outer skirt 142, respectively), the remainder of the outer surface of the each of the valve leaflets 134 may be free from direct contact with the expandable framework 132 as the plurality of valve leaflets 134 opens and/or closes. Specifically, while a portion of the free edge of each of the plurality of valve leaflets 134 and the root edge of each of the plurality of valve leaflets 134 may be coupled to a portion of the expandable framework 132 (e.g. the commissures 146 and the lower edge of the outer skirt 142, respectively), the remainder of the outer surface of the each of the valve leaflets 134 may be free from direct contact with the interleaflet struts 144a/144b/147a/147b/149a/149b defining the interleaflet triangle regions of the expandable framework 132 as the plurality of valve leaflets 134 opens and/or closes.

As described herein and illustrated in FIG. 4, the replacement heart valve implant 130 may include an outer skirt 142. In some embodiments, the outer skirt 142 may include an upper edge 150 (e.g., distal edge) and a lower edge 166 (e.g., proximal edge). Additionally, the outer skirt 142 may extend circumferentially around a portion of the abluminal surface (e.g., outer facing surface) of the expandable framework 132 and define a substantially tubular shape. In some examples, the “height” of the outer skirt 142 (e.g., the length of the outer skirt 142 form the lower edge 166 to the upper edge 150) may be about 1-20 mm, about 2-14 mm, about, 3-12 mm, about 4-10 mm, or about 5-8 mm.

In some embodiments, the outer skirt 142 may be disposed at and/or adjacent a portion of the struts defining the lower crown 136. It can be appreciated that the outer skirt 142 may be disposed between the expandable framework 132 and the vessel wall in order to prevent fluid, such as blood, from flowing around the replacement heart valve implant 130 and/or the expandable framework 132 in a downstream direction. In other words, the outer skirt 142 may be designed to fill with fluid, thereby sealing against the vessel wall to ensure that fluid is directed through the replacement heart valve implant 130. As will be described in great detail below, in some examples, fluid flowing from the aorta 152 toward the left ventricle 154 (after the left ventricle relaxes) may contact the abluminal side of each of the plurality of valve leaflets 134. Further, this fluid flow may exert a force which pushes each of the plurality of valve leaflets 134 inward (e.g., toward the central longitudinal axis of the implant 130), thereby causing the closing and coaptation action of the plurality of valve leaflets 134 (to prevent further retrograde fluid flow). Further yet, this fluid flow may also be simultaneously directed outwards, thereby causing the outer skirt 142 to expand and seal against the native valve annulus. The simultaneous coaptation (e.g., closing) of the valve leaflets 134 with expansion and sealing of the outer skirt 142 may be referred to as synchronized coaptation and sealing. As discussed herein, when fully closed, the heart valve leaflets 134 may prevent the retrograde flow of fluid from the aorta 152 into the left ventricle 154.

The detailed view of FIG. 4 illustrates that, in some examples, the root edge 135 of each of the plurality of valve leaflets 134 may be attached to the lower edge 166 of the outer skirt 142. The detailed view of FIG. 4 illustrates that the root edge 135 of each of the plurality of valve leaflets 134 may be sewn (e.g., sutured, stitched, etc.) to the lower edge 166 of the outer skirt 142. It can be appreciated that the stitch 164 may extend circumferentially around the expandable framework 132. For example, the detailed view of FIG. 4 illustrates a continuous stitch 164 passing through both the root edge 135 of each of the plurality of valve leaflets 134 and the lower edge 166 of the outer skirt 142, thereby attaching the root edge 135 of each of the valve leaflets 134 with the lower edge 166 of the outer skirt 142.

In some examples, the stitch 164 may include a whip stitch that attaches the root end 135 of each of the plurality of valve leaflets 134 with one another to form a tight cylinder (e.g., the stitch 164 extends circumferentially around the expandable framework 132, thereby substantially preventing fluid from leaking through the seam created by the stitch 164). In some examples, the replacement heart valve implant 130 may further include an additional stitch positioned distal (e.g., upstream) of the stitch 164, whereby this additional stitch extends circumferentially around the expandable framework 132, thereby providing an additional stitched seam to prevent fluid leakage and prevent the valve leaflets 134 from stretching.

Further, as illustrated in the detailed view of FIG. 4, it can be appreciated that the stitch 164 may pass through the material defining the valve leaflets 134 and the lower edge of the outer skirt 142 when attaching the valve leaflets 134 to the lower edge of the outer skirt 142, thereby creating a gap between the material of the valve leaflet 134 and the outer skirt 142 through which the struts of the expandable framework 132 may extend. In other words, the stitch 164 may bind the valve leaflets 134 and the outer skirt 142 together such that the stitch 164 straddles and loops around the expandable frame 132, thereby attaching the valve leaflets 134, the outer skirt 142 and the stitch 164 to the expandable frame 132. Further, in some instances, the stitch 164 may also wrap around the expandable frame 132 to secure the valve leaflets 134, the outer skirt 142 and the stitch 164 to the expandable frame 132.

Additionally, in some embodiments, the outer skirt 142 may be coupled to a portion of the expandable framework 132 just below the upper crown 138 and/or a portion of an adjacent valve leaflet 134, respectively. For example, FIG. 4 illustrates that the upper edge 150 of the outer skirt 142 may be coupled to the expandable framework 132 just below the upper crown 138 and/or a valve leaflet 134 at a plurality of attachment points 148. In some instances, the upper edge 150 of the outer skirt 142 may be sutured to the upper crown 138 and/or a valve leaflet 134 at the plurality of attachment points 148. The attachment points 148 may be spaced around the circumference of the expandable framework 132. For example, in some instances, the valve 130 may include 2, 3, 4, 5, 6, 7, 8, 9, 10, 11, 12, 13, 14, 15, 16 or more attachment points 148 spaced around the circumference of the expandable framework 132.

It can be appreciated that attachment of the outer skirt 142 to the expandable framework 132 at a location just below the upper crown 138 may enable optimal filling, expansion and pressurization of the outer skirt as fluid flows from the aorta toward the ventricle (after the ventricle relaxes), as described above. It can be appreciated that positioning the outer skirt 142 just below the upper crown 138 may allow the outer skirt to contact and seal against the native annulus by preventing interference with native leaflets as the upper crowns push aside the native valve leaflets.

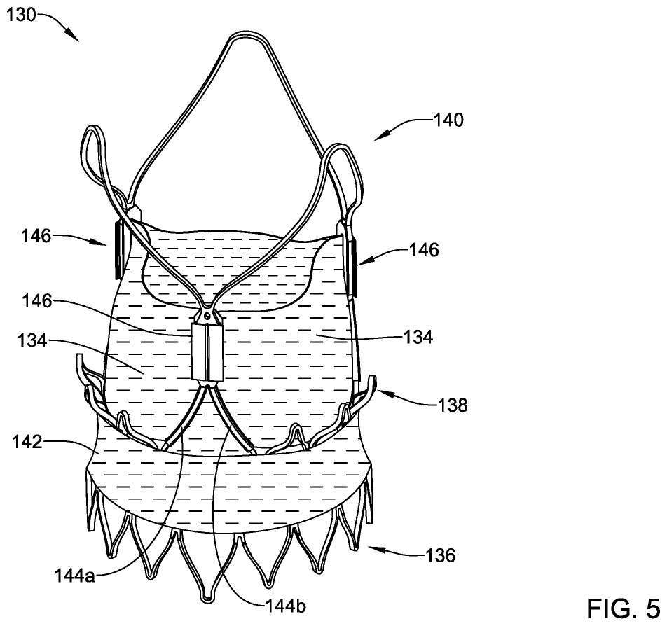

As will be further described with respect to FIG. 5 below, it can be appreciated that the portions of the upper edge 150 of the outer skirt 142 which are not attached to the expandable framework 132 and/or a valve leaflet 134 are free from both the expandable framework 132 and/or the valve leaflets 134. In other words, the unattached portions of the upper edge 150 of the outer skirt 142 is free to expand, flex, etc., thereby permitting fluid (e.g., blood) to flow into and fill the outer skirt 142 (e.g., the fluid flows into the space defined by an outer surface of the valve leaflets 134 and the inner surface of the outer skirt 142). In some instances, the outer skirt 142 may resemble a tubular-shaped pocket extending circumferentially around the outer surface of the expandable framework 132. It can be appreciated that designing the valve 130 to include the upper edge 150 of the outer skirt 142 to include a combination of attached (e.g., via attachment points 148) and unattached portions enables optimal filling, expansion and pressurization of the outer skirt 142 to seal against the native annulus as fluid flows from the aorta toward the ventricle (after the ventricle relaxes).

In some embodiments, the outer skirt 142 may include a polymer, such as a thermoplastic polymer. In some embodiments, the outer skirt 142 may include at least 50 percent by weight of a polymer. In some embodiments, the outer skirt may include a polymer, such as a thermoplastic polymer. In some embodiments, the outer skirt 142 may include at least 50 percent by weight of a polymer. In some embodiments, one or more of the plurality of valve leaflets 134, the outer skirt 142, or both the plurality of valve leaflets 134 and the outer skirt 142 may be formed of the same tissue, polymer or combination of tissue and polymers. In some embodiments, the polymer may be a polyurethane. Some suitable but non-limiting examples of materials that may be used to form the plurality of valve leaflets 134, the outer skirt 142, or both the plurality of valve leaflets 134 and the outer skirt 142, may include but are not limited to polymers, composites, and the like, are described herein.

A described herein, the outer skirt 142 may be formed from a highly flexible and/or compliant thin film material. In some instances, the outer skirt 142 material may be designed such that it may extend to 25-50 percent of the native valve diameter at 120 mmHg. In some embodiments, the outer skirt 142 may be configured to conform to the annulus of the native heart valve. For example, the material utilized to construct the outer skirt 142 may be configured to occupy space between the expandable framework 132 and the annulus of the native heart valve. In some embodiments, the material utilized to construct the outer skirt 142 may be configured to conform to stenosis, calcification, calcium nodules, etc. associated with the native heart valve and/or surrounding tissue(s). In some patients, stenosis, calcification, calcium nodules, etc. may cause the native heart valve to have or assume an irregular shape, such that deployment of the replacement heart valve implant 130 may not obtain sufficient sealing around the expandable framework 132 due to gaps and/or space left between the expandable framework 132 and the annulus of the native heart valve and/or surrounding tissue(s). The outer skirt 142 of the replacement heart valve implant 130 of the current disclosure may be designed to fill those gaps and/or space(s) during diastole by preventing fluid or blood from passing around the replacement heart valve implant 130 and back into the patient's heart. It can be appreciated that at the point of maximum arterial pressure during the heart's pumping cycle, the valve leaflets 134 close. Accordingly, the risk of leakage around the valve 130 is highest at this moment and the increased pressure may cause leak channels to form. The design of the valve 130 of the current disclosure utilizes the pressure increase (e.g., higher pressures) to progressively expand the outer skirt against the annulus wall, thereby preventing leak channels.

As described herein, FIG. 4 illustrates the replacement heart valve implant 130 in a closed configuration. It can be appreciated that this configuration may illustrate a configuration in which the heart has relaxed after pumping fluid from the left ventricle 154 into the aorta 152 (e.g., a configuration of the valve implant 130 at the end of systole). Accordingly, after being pumped into the aorta 152, the fluid in aorta 152 may attempt to flow back into the left ventricle 154, thereby applying back pressure onto portions of the replacement heart valve implant 130. As discussed herein, the fluid may apply back pressure onto the plurality of heart valve leaflets 134. This back pressure may force the valve leaflets 134 to coapt with one another, whereby the free edges of each of the heart valve leaflets 134 seal against one another to prevent fluid from flowing in a retrograde manner into the left ventricle 154. It can be appreciated that the material forming the valve leaflets 134 may extend away from the inner surface of the expandable framework 132. Specifically, the material forming the valve leaflets 134 may extend away from the struts defining the interleaflet triangles, as described herein.

Additionally, as described herein, retrograde fluid flow may also flow into the space defined by the inner surface of the outer skirt 142 and the outer (e.g., abluminal) surface of the valve leaflets 134. It can be appreciated that as the fluid flows into this space (e.g., the tubular pocket defined by the outer skirt 142), the outer skirt 142 may capture fluid, expand radially outward and seal against the annulus, thereby preventing fluid from leaking back into the left ventricle 154. It can be appreciated that this expansion may offer a dicrotic notch effect (e.g., a system compliance) that promotes leaflet closure in the same way as the aorta expands in healthy patient anatomies. High risk patients may often exhibit poor aortic compliance (e.g., stiff, hard aortas) and, therefore, designing a replacement heart valve implant (such as the implant 130 disclosed herein) to include an expandable outer skirt addresses this issue by promoting dicrotic notch pressurization, leaflet coaptation and improved valve closure.

Further, it can be appreciated that, in the configuration shown in FIG. 4, because the outer skirt 142 is attached to the plurality of valve leaflets 134, they may be pressured in tandem with one another, thereby ensuring that the outer skirt 142 rapidly expands outward to cause its outer surface to seal against the vessel wall, thereby preventing leak channels/pathways. In other words, the outer skirt 142 may be configured to extend radially outward from the expandable framework 132 to prevent leak channel formation around an exterior of the expandable framework 132. The outer skirt 142 thereby prevents fluid flow and/or aortic jetting through the native heart valve and back into the patient's heart (e.g., back into the left ventricle). As such, the outer skirt 142 may be configured to prevent and/or substantially reduce paravalvular leakage around the expandable framework 132 during diastole.

FIG. 5 illustrates that the example replacement heart valve implant 130 described in FIG. 4 in an open configuration. For example, FIG. 5 illustrates the valve implant 130 in a configuration in which the free edge of each of the valve leaflets 134 have moved apart from one another. It can be appreciated that, in this configuration, fluid is permitted to flow downstream through the valve leaflets 134. FIG. 5 illustrates that as fluid flows through the valve leaflets 134, the outer surface of the leaflets 134 may be permitted expand radially outward, towards the inner surface of the expandable framework 132. For example, FIG. 5 illustrates the valve leaflets 134 expanding radially outward such that the outer surface of the leaflets 134 move towards the inner surface of the interleaflet struts 144a/144b. FIG. 5 further illustrates that as the fluid flows through the replacement heart valve implant 130, the outer skirt 142 is depressurized. In some instances, fluid (e.g., blood) can flow both through and around the valve 130. Blood flow around the valve 130 may promote wash-out and reduce the risk of stagnation. This may reduce the potential for the expandable frame 132 to shift.

The materials that can be used for the various components of the medical device system and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the expandable framework, the inner skirt, the outer skirt, the plurality of leaflets, the plurality of pockets, the plurality of biasing arms, and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is defined in the language in which the appended claims are expressed.

What is claimed is:

1. A heart valve, comprising:
an expandable framework configured to shift from a closed configuration to an expanded configuration, the expandable framework having an inflow end, an outflow end and a lumen extending therein;
a plurality of valve leaflets coupled to the expandable framework, each of the plurality of valve leaflets including a root edge and a free edge; and
an outer skirt coupled to the plurality of valve leaflets, the outer skirt including a proximal edge;
wherein the root edge of each of the plurality of valve leaflets is attached to the proximal edge of the outer skirt.

2. The heart valve implant of claim 1, wherein the outer skirt is positioned along an outer surface of the expandable framework.

3. The heart valve implant of claim 2, wherein the plurality of valve leaflets is positioned within the lumen of the expandable framework.

4. The heart valve implant of claim 3, wherein the root edge of each of the plurality of valve leaflets is sutured to the proximal edge of the outer skirt.

5. The heart valve implant of claim 4, wherein root edge of each of the plurality of valve leaflets and the proximal edge of the outer skirt are sutured around the expandable framework such that the root edge of the plurality of valve leaflets and the proximal edge of the outer skirt may move relative to the expandable framework.

6. The heart valve implant of claim 1, wherein the outer skirt further includes a distal edge, and wherein the distal edge of the outer skirt is attached to one or more of the plurality of valve leaflets at one or more attachment points.

7. The heart valve implant of claim 1, wherein the outer skirt extends radially outward from the expandable framework to capture fluid flow around an exterior of the expandable framework within the outer skirt.

8. The heart valve implant of claim 1, wherein the outer skirt is substantially impervious to fluid.

9. The heart valve implant of claim 1, wherein the proximal edge of the outer skirt is disposed upstream of the inflow end of the expandable framework.

10. The heart valve implant of claim 9, wherein the expandable framework further includes an upper crown, wherein the distal edge of the outer skirt is disposed proximal of the upper crown.

11. A heart valve, comprising:
an expandable framework having an inflow end, an outflow end, and a medial region positioned between the inflow end and the outflow end;
a plurality of valve leaflets attached to the medial region of the expandable framework, each of the plurality of valve leaflets including a root edge; and
an outer skirt disposed along an outer surface of the expandable framework, the outer skirt including a proximal edge and a distal edge;
wherein the outer skirt is configured to shift between an unexpanded configuration and an expanded configuration;
wherein the root edge of each of the plurality of valve leaflets is attached to the proximal edge of the outer skirt.

12. The heart valve implant of claim 11, wherein the plurality of valve leaflets is positioned within a lumen of the expandable framework.

13. The heart valve implant of claim 11, wherein the root edge of each of the plurality of valve leaflets is sutured to the proximal edge of the outer skirt.

14. The heart valve implant of claim 13, wherein root edge of each of the plurality of valve leaflets and the proximal edge of the outer skirt are sutured around the expandable framework such that the root edge of the plurality of valve leaflets and the proximal edge of the outer skirt may move relative to the expandable framework.

15. The heart valve implant of claim 11, wherein the distal edge of the outer skirt is attached to one or more of the plurality of valve leaflets at one or more attachment points.

16. The heart valve implant of claim 11, wherein the outer skirt is configured to capture fluid flow around an exterior of the expandable framework within the outer skirt.

17. The heart valve implant of claim 11, wherein the outer skirt is substantially impervious to fluid.

18. The heart valve implant of claim 11, wherein the proximal edge of the outer skirt is disposed upstream of the inflow end of the expandable framework.

19. The heart valve implant of claim 11, wherein the expandable framework further includes an upper crown, wherein the distal edge of the outer skirt is disposed proximal of the upper crown.

20. A method for delivering a heart valve implant at a target site, the method comprising:
advancing a heart valve implant to a target site adjacent the heart, the heart valve implant including:
an expandable framework configured to shift from a closed configuration to an expanded configuration, the expandable framework having an inflow end, an outflow end and a lumen extending therein;
a plurality of valve leaflets coupled to the expandable framework, each of the plurality of valve leaflets including a root edge and a free edge; and
an outer skirt coupled to the plurality of valve leaflets, the outer skirt including a proximal edge;
wherein the root edge of each of the plurality of valve leaflets is attached to the proximal edge of the outer skirt; and
deploying the implantable heart valve at the target site.
